(19) United States
(12) Patent Application Publication
(10) Pub. No.: US 2022/0048958 A1
(43) Pub. Date: Feb. 17, 2022

(54) USE OF VARIOVORAX MICROBES AS AN ALTERNATIVE TREATMENT FOR COCCIDIOSIS

(71) Applicant: ZIVO Bioscience, Inc., Keego Harbor, MI (US)

(72) Inventors: Andrew A. Dahl, Keego Harbor, MI (US); William P. Pfund, Keego Harbor, MI (US); Amy E. Steffek, Keego Harbor, MI (US)

(73) Assignee: ZIVO Bioscience, Inc., Keego Harbor, MI (US)

(21) Appl. No.: 17/400,790

(22) Filed: Aug. 12, 2021

Related U.S. Application Data
(60) Provisional application No. 63/064,706, filed on Aug. 12, 2020.

(51) Int. Cl. C07K 14/32; A23K 10/16

(52) U.S. Cl. CPC C07K 14/32; A23K 10/16

(57) ABSTRACT

Compounds capable of selectively modulating the TLR signaling pathway provide an improved treatment method for a broad variety of diseases in both animals and humans. The mechanisms of action in the treatment and/or prevention of coccidiosis and other conditions related to gut inflammation are via a direct effect on innate and adaptive immune pathways. The downstream results are improvements in performance parameters related to gut health (including altering gut microbes, conversion rates, and body weight gains among others. When administered to animals, the bioactives of the disclosed inventive compound mitigate the effects of coccidiosis via an enhanced immune response rather than a direct effect on parasites, such as the Eimeria parasite. The mechanisms of action of the disclosed inventive compound and method are via immune system priming rather than a direct effect on pathogens, thus there is no risk of treatment resistance being developed.

### CROSS REFERENCE TO RELATED APPLICATION

This application is a US. Non-provisional Patent Application of U.S. Provisional Patent Application No. 63/064,706, entitled “Use of Variovorax Microbes as a Coccidiostat,” filed Aug. 12, 2020, which is herein incorporated by reference in its entirety for all purposes.

### TECHNICAL FIELD

The present invention relates to the use of a bacteria-based compound in the prevention and treatment of disease. More particularly, the present invention relates to a compound and the use of a compound such as that derived from a lipopolysaccharide (LPS) of Gram-negative bacteria that selectively modulates the Toll-like receptor (TLR) pathway in the prevention and treatment of disease in both animals and humans.

### BACKGROUND OF THE INVENTION

Lipopolysaccharide (LPS), also known as endotoxin, is a major component of the outer membrane of Gram-negative bacteria. The lipid A moiety of LPS is responsible for most of the toxicity of Gram-negative bacteria. Some LPS compounds are known to interact with and activate Toll-like receptor 4 (TLR4). Activation of TLR4 results in inflammatory cytokine production and activation of the innate immune system. TLR activation via the recognition of pathogenic organisms is a crucial step in the innate immune response. Aberrant activation of this defense mechanism, however, can lead to non-specific inflammatory responses and perpetuate autoimmune reactions.

TLR4, found on immune system cells throughout the body, as well as on other cell types, such as heart, liver, and fat cells, recently emerged as a common factor linking diet, gut microbiota, and metabolic health.

Whether activated through infection or inflammation, TLR4 is involved in a multitude of acute and chronic diseases in humans and animals. TLR4 is an attractive target for non-antibiotic therapies in poultry production where parasite-induced inflammation, such as the Eimeria parasite-induced inflammation, can compromise gut integrity resulting in decreased nutrient utilization and impaired growth. Additionally, genetic differences conferring a less active TLR4 has been linked to increased resistance to Salmonella infection in chickens. TLR4 has been implicated in chronic back pain and disc degeneration and plays a role in the pathophysiology of osteoarthritis, which is the eighth leading cause of human disability globally and also affects more than 60% of canines above the age of 7. As such, the investigation of TLR4 as a high value therapeutic target for an array of disorders is ongoing.

Several examples of inflammatory and autoimmune conditions linked to TLR4 are known. These examples include, but are not limited to, sepsis, lupus, multiple sclerosis, inflammatory bowel disease, rheumatoid arthritis, psoriasis, asthma, allergies, neurodegeneration and CNS diseases linked to neuroinflammation, cancer, viral infection, amyotrophic lateral sclerosis (ALS), neuropathic pain, diabetes-related complications (such as diabetic nephropathy, diabetic retinopathy and diabetic neuropathy), COPD, pathogenesis of many cardiovascular diseases (including atherosclerosis, hypertension, and stroke), obesity-associated metabolic inflammation, drug abuse, major depressive disorder, and nonalcoholic steatohepatitis (NASH).

A number of TLR4 modulators have been evaluated in human clinical trials. For example, the TLR4 inhibitors Eritoran and Resatorvid, potential candidates for the treatment of severe sepsis, a condition more deadly than breast, colon, and lung cancer combined, showed promise in early human clinical trials. However, these compounds failed to reduce mortality in Phase 3 clinical trials. Likewise, a potential TLR4 inhibitor for rheumatoid arthritis, a condition for which an estimated 860 individuals out of every 100,000 in the U.S. suffer, showed early promise yet failed to show efficacy in Phase 2 clinical trials. A Phase 2 Eritoran trial to reduce inflammation and improve glucose metabolism in insulin resistant obese patients with Type 2 Diabetes was terminated in 2018. A recently completed Phase 2 multiple sclerosis trial demonstrated that Ibudilast, a TLR4 antagonist, was associated with slower progression of brain atrophy compared to placebo but had higher rates of gastrointestinal side effects, headache, and depression. There are several candidates, including JKB-122 for autoimmune hepatitis and Ibudilast for ALS for which there are active ongoing clinical trials. Additionally, candidates that have demonstrated suboptimal therapy in one disease may still be assessed for use in other conditions linked to excessive TLR4 signaling.

### SUMMARY OF THE INVENTION

The disclosed inventive concept provides an improved treatment method for a broad variety of diseases in both animals and humans. The mechanisms of action in the treatment and/or prevention of coccidiosis, necrotic enteritis, and other conditions related to gut inflammation are via a direct effect on innate and adaptive immune pathways. Reference may be made to Applicants' co-owned and pending application U.S. Ser. No. 17/358,878, filed Jun. 25, 2021, for “Immune Priming to Accelerate/Enhance Immune Response Through Administration of Natural Immune Modulator,” which is herein incorporated by reference in its entirety for all purposes.

Outbreaks of coccidiosis and necrotic enteritis are typically concurrent. It is known that a coccidiosis infection predisposes an animal to necrotic enteritis as the damage from the coccidiosis infection creates an ideal environment in which the coccidia parasite may flourish. The downstream results are improvements in performance parameters related to gut health (including altering gut microbes), feed conversion rates, and body weight gains among others. When administered in poultry feed or drinking water, the bioactives of the disclosed inventive compound mitigate the effects of coccidiosis via an enhanced immune response rather than a direct effect on a parasite, such as the Eimeria parasite.

The mechanisms of action of the disclosed inventive compound and method are via immune system priming rather than a direct effect on pathogens, thus there is no risk of treatment resistance being developed. Due to broad spectrum immune modulation, animal growth and development are enhanced systemically.

### DETAILED DESCRIPTION OF THE PREFERRED EMBODIMENT

In the following description, various operating parameters and components are described for different constructed embodiments. These specific parameters and components are included as examples and are not meant to be limiting. Unless otherwise noted, all technical and scientific terms used herein are to be accorded their common meanings as would be understood by one having ordinary skill in the art.

The Compound Used in Treatment

The disclosed method of treatment preferably, but not exclusively, utilizes a compound generally derived from a lipopolysaccharide (LPS) of Gram-negative bacteria. By administering the compound early in broiler life, disease prevention and treatment via immune modulation are achieved. As used herein, the term “inhibitor” refers to a molecule that reduces or attenuates the activity induced by another molecule, receptor, cellular structure, or organ. By way of example, a compound that might block the LPS-dependent activation of TLR4 present on the surface of a host immune cell would be regarded as an inhibitor of this particular pathway.

As used herein, “modulator” refers to an activator, an inhibitor, or both. Modulation may be the result of activity by at least one Toll-like receptor (TLR), such as TLR4 or possibly TLR2. As used herein, the term “inhibitor” refers to a molecule that reduces or attenuates the activity induced by another molecule. By way of example, a compound that might block the LPS-dependent activation of TLRs present on the surface of immune cells in humans and animals would be regarded as an inhibitor of this particular pathway.

As used herein, the term “algal culture” is defined as an algal organism and bacteria (one or more types) that grow together in a liquid medium. Unless expressly stated otherwise, the term “algal biomass” refers to the algal cells and bacterial cells (with the liquid culture medium removed). The “algal biomass” can be wet material or dried material.

Unless expressly stated otherwise, the term “algal supernatant” is defined as the culture medium in which the algal biomass is grown that contains excreted compounds from the algal biomass. Algal supernatant is obtained by growing algal biomass in culture medium for an appropriate length of time and then removing the algal and bacterial cells by filtration and/or centrifugation.

It is known that bacteria of the Variovorax genus and the Rhodobacter genus are metabolically versatile. Variovorax is a Gram-negative aerobic bacterium that can grow under a variety of conditions. It is part of the subclass Proteobacteria and is capable of metabolically utilizing several natural compounds generated by plants or algae. Rhodobacter can grow under a broad variety of conditions, including both photosynthesis and chemosynthesis. Growth can also be achieved under both anaerobic and aerobic conditions. Rhodobacter sphaeroides represents a Gram-negative facultative bacterium and is a member of the α-3 subdivision of the Proteobacteria.

Embodiments of the compound used in the treatment of disease as set forth herein include one or more LPS/Lipid A compounds produced by Gram-negative bacterial strains for use as selective modulators of the TLR4 signaling pathway. The disclosed inventive concept involves any combination of three fundamental steps: (1) the Gram-negative bacteria produces LPS/Lipid A compounds; (2) the LPS/Lipid compounds modulate TLR4 activity through activation or inhibition; and (3) a downstream effect results in reduced negative impact to health and growth due to coccidiosis, necrotic enteritis, and other conditions related to gut inflammation.

In an embodiment, the LPS/Lipid A compounds used as selective modulators of the TLR4 signaling pathway are produced from a Variovorax paradoxus strain. The Variovorax paradoxus strain may be a naturally occurring strain found in an algal biomass. (As noted above in Paragraph [0015], biologically active by-products [including either excreted products or structural components] may be found in the algal supernatant.) The algal biomass may comprise the algal species Klebsormidium flaccidum. More specifically, the algal biomass culture may comprise the algal strain Klebsormidium flaccidum, var. ZIVO.

In another embodiment, the LPS/Lipid A compounds used as selective modulators of the TLR4 signaling pathway are produced from a Rhodobacter sphaeroides strain. Extensive studies have been undertaken regarding the structure and function of Rhodobacter sphaeroides. More focused studies have examined the photosynthetic characteristics of Rhodobacter sphaeroides. While it is known that in human cells lipopolysaccharides from Rhodobacter sphaeroides are effective TLR4 antagonists that prevent TLR4-mediated inflammation by blocking LPS/TLR4 signaling, the inventors employed a testing methodology to address multiple live growth performance parameters in poultry to arrive at the conclusion that an LPS compound derived from Rhodobacter sphaeroides proved effective as an alternative treatment to coccidiosis. Research further showed that combining a compound that acts as TLR4 inhibitor in human in vitro assays with an activator of TLR2 (such as LPS from Gram-negative bacteria) provides an anti-coccidiosis effect.

Accordingly, embodiments of the compound used in the treatment of disease according to the present disclosure are directed to one or more LPS/Lipid A compounds produced by a Gram-negative bacterial strain of the group Variovorax or the group Rhodobacter for use as selective modulators of the TLR signaling pathway. A specific embodiment of the disclosed inventive concept is directed to the use of an LPS/Lipid A compound used as a selective modulator of the TLR4 signaling pathway produced from the Variovorax paradoxus strain and the Rhodobacter sphaeroides strain.

The LPS/Lipid A compound employed herein may be obtained from the Variovorax paradoxus strain and/or the Rhodobacter sphaeroides strain by any suitable method, but in specific embodiments they are extracted using standard multi-step LPS extraction protocols, such as: (1) extracting freeze-dried bacteria with a solution of phenol/guanidine thiocyanate and collecting the water layer for freeze-drying; (2) resolubilizing the freeze-dried fraction in water; (3) ultrafiltration of the solubilized fraction to remove low molecular weight substances and salts; (4) affinity purifying the high-molecular weight fraction using a polymyxin B resin column such as Affi-prep polymyxin matrix material (Bio-Rad), from which an active fraction is eluted with 1 deoxycholate and, optionally; (5) performing additional purification using size-exclusion chromatography.

In some examples, multiple types of LPS extraction protocols are employed to obtain an LPS compound from the bacteria, and extraction procedures may be performed more than once. Once the LPS compound is extracted and purified from the bacteria, the Lipid A fraction may be prepared by acid hydrolysis or other suitable technique.

In some examples, analysis of the structure of the LPS compound is performed using routine methods in the art, including using mass spectrometry, gas chromatography, or both. As a non-limiting example, in one embodiment results of liquid chromatography analysis of the LPS isolated from the Varivorax paradoxus strain showed the presence of both hydroxyl-decanoic and hydroxyl-octanoic fatty acids on the lipid A moiety. By way of a further non-limiting example, in another embodiment results of gas chromatography-mass spectrometry (GC-MS) analysis of the LPS isolated from the Variovorax paradoxus strain showed that the main saturated fatty acid is lauric acid, with one or two molecules per lipid A structure.

One or more LPS/Lipid A compounds derived from Gram-negative bacterial strains, such as Variovorax paradoxus or Rhodobacter sphaeroides, may selectively modulate the TLR4 signaling pathway to alter inflammatory responses and to improve immune health in a variety of uses and applications. In an embodiment, the LPS/Lipid A compound derived from Variovorax paradoxus or Rhodobacter sphaeroides may be incorporated within an algal-based feed ingredient to improve gut health of poultry.

The disclosed LPS/Lipid A compound derived from Variovorax paradoxus or Rhodobacter sphaeroides may be used to improve the health of poultry through a variety of mechanisms. For example, the LPS/Lipid A compound may protect against internal inflammation in poultry by negatively regulating inflammatory mediators via the downregulation of TLR4 expression and the downstream inhibition of NF-kappa B activation in a typical inflammatory cascade. In another example, the LPS/Lipid A compound may inhibit the activation of TLR4 in poultry by interfering with cysteine residue-mediated receptor dimerization. In yet another example, the LPS/Lipid A compound may inhibit the ability of non-infectious and infectious stimuli to interact with TLR4 and trigger a pro-inflammatory response, thereby improving poultry gut integrity. In a further example, the LPS/Lipid A compound may modulate TLR4 through either ligand-dependent or ligand-independent activation. As another example, the LPS/Lipid A compound may act in concert with other TLR agonists to provide a heightened immune response, while reducing the metabolic costs to the host.

When including the disclosed inventive concept as bacterial biomass in animal feed, the combined batch is preferably provided in an amount of between about 20.0 g composition to ton of finished feed to about 250.0 g composition to ton of finished feed, is more preferably provided in an amount of between about 125.0 g composition to ton finished feed to about 175.0 g composition to ton of finished feed, and is most preferably provided in an amount of between about 100.0 g composition per ton of finished feed to about 150.0 g composition per ton of finished feed. The ideal suggested and non-limiting ratio is about 125.0 g composition per ton of finished feed for maximum effect.

Study No. 1

The first study to demonstrate the effectiveness of the compound of the disclosed inventive concept and a treatment method was designed to identify the comparative anticoccidial efficacy of the inventive compound administered in feed to animals, particularly chickens, and more specifically, broiler chickens.

Study No. 1—Treatment Method

A non-limiting example of a method for the improvement of growth efficiency in broiler chickens exposed to a coccidiosis disease challenge is set forth. It is to be understood that the following method is not intended as being the sole treatment method but is only exemplary. The study compared four treatment regimens: (1) no additive material in the feed, no coccidiosis challenge in the poultry; (2) no additive material in the feed, coccidiosis challenge in the poultry; (3) Salinomycin in the feed, coccidiosis challenge in the poultry; and (4) the inventive compound in the feed, coccidiosis challenge in the poultry. Salinomycin, a polyether ionophore antibiotic isolated from Streptomyces albus, is commonly used as an antibiotic in the treatment of coccidiosis.

The study animals were chickens, specifically male broiler chickens. Day of hatch male broiler chicks were obtained. At the hatchery, the birds were sexed. Sets of ten chicks were randomly selected, group weighed and placed into cages. The number and disposition of all birds not used for allocation were documented. No birds were replaced during the course of the study. There were 80 birds per treatment group.

The poultry cages were blocked by location in the battery with block size equal to treatments. The study began when the birds, specifically broiler chickens, were placed on the day of hatch (DOT 0) at which time they were allocated to the experimental cages. Only healthy birds were selected. At placement the birds were fed the treatment feeds. Thermostatically controlled gas furnace/air conditioner maintained uniform temperature in the cages.

An un-medicated commercial type chicken starter compounded with feedstuffs commonly used in the United States was formulated. The diet was an all-vegetable feed having no antibiotics, organic acid, NSP enzyme, or direct fed microbial. This ration was fed ad libitum from the date of chick arrival until Day 28 of the study. The diet formulation was included in the source data. Experimental treatment feeds were prepared from this basal starter feed. Quantities of all basal feed and test articles used to prepare treatment batches were documented.

One each from the beginning, middle, and end of each batch of treatment diet were collected and mixed to form a composite sample. One composite sample was taken from the composite for each treatment for analysis.

All birds were weighed by cage on DOT 0, 14, 20 and 28. Feed was weighed in on DOT 0. The remaining feed was weighed on DOT 14, 20, and 28.

Coccidial oocysts inoculation procedures were carried out. On Day 14 of the study, all birds except T1 orally received a mixed E. acervulina, E. maxima, and E. tenella coccidial inoculum diluted to a 1.0 ml volume (p.o.). Inoculum level attempted to produce a moderate challenge.

On DOT 20, a select number of birds from each cage were selected, sacrificed, weighed, and examined for the degree of presence of coccidia lesions. The Johnson and Reid (1970) method of coccidiosis lesion scoring was used to score the infected region(s) of the intestine. The scoring was based on a 0 to 4 score, with 0 being normal and 4 being the most severe. Also on Day 20 mixed feces were collected from each cage. Each sample was examined for the number of oocysts per gram fecal material by fecal floatation.

Study No. 1—Data

The collected data are set forth in the following chart.

| DAY 14 | Feed Intake | FCR | Wt. Gain | |
|---|---|---|---|---|
| 1. No Additive, Non-Infected | 2.310a | 1.155a | 0.217a | |
| 2. No Additive, Infected | 2.461a | 1.142a | 0.230a | |
| 3. Salinomycin, 60 g/t | 2.384a | 1.156a | 0.218a | |
| 4. Inventive Compound, 0.551% | 2.709a | 1.150a | 0.246a | |
| DAY 0-20 | Feed Intake | FCR | Wt. Gain | |
| 1. No Additive, Non-Infected | 5.487a | 1.497c | 0.401a | |
| 2. No Additive, Infected | 4.595b | 1.768a | 0.287c | |
| 3. Salinomycin, 60 g/t | 5.433a | 1.597bc | 0.360b | |
| 4. Inventive Compound, 0.551% | 4.845ab | 1.647ab | 0.307c | |
| DAY 14-20 | Feed Intake | FCR | Wt. Gain | |
| 1. No Additive, Non-Infected | 3.177a | 1.502c | 0.227a | |
| 2. No Additive, Infected | 2.134a | 2.235a | 0.100c | |
| 3. Salinomycin, 60 g/t | 3.049a | 1.744b | 0.185b | |
| 4. Inventive Compound, 0.551% | 2.137a | 2.112a | 0.104c | |
| DAY 0-28 | Feed Intake | FCR | Wt. Gain | |
| 1. No Additive, Non-Infected | 5.482a | 1.554c | 0.481a | |
| 2. No Additive, Infected | 5.690a | 2.086a | 0.289c | |
| 3. Salinomycin, 60 g/t | 5.918a | 1.625bc | 0.376b | |
| 4. Inventive Compound, 0.551% | 5.520a | 1.766b | 0.343bc | |
| DAY 14-28 | Feed Intake | FCR | Wt. Gain | |
| 1. No Additive, Non-Infected | 3.172ab | 1.612c | 0.306a | |
| 2. No Additive, Infected | 3.229ab | 3.132a | 0.102c | |
| 3. Salinomycin, 60 g/t | 3.534a | 1.766c | 0.202b | |
| 4. Inventive Compound, 0.551% | 2.812b | 2.380b | 0.140c | |
| | Eimeria | Eimeria | Eimeria | |
| Lesion Scores | acervulina | maxima | tenella | Avg. |
| 1. No Additive, Non-Infected | 0.0d | 0.0d | 0.0d | 0.0d |
| 2. No Additive, Infected | 3.0a | 2.5a | s.8a | 2.8a |
| 3. Salinomycin, 60 g/t | 2.1c | 0.9c | 0.7c | 1.3c |
| 4. Inventive Compound, 0.551% | 2.6b | 2.0b | 2.3b | 2.3b |
| | Eimeria | Eimeria | Eimeria | |
| Oocysts per Gram Fecal Material | acervulina | tenella | maxima | Avg. |
| 1. No Additive, Non-Infected | 0c | 0c | 0b | 0b |
| 2. No Additive, Infected | 57112a | 23153a | 2701a | 82966a |
| 3. Salinomycin, 60 g/t | 4511a | 4727c | 484b | 9722b |
| 4. Inventive Compound, 0.551% | 46840a | 20835a | 2159a | 69835a |

The letters shown with each result denote statistical significance—those with the same letters are not statistically different from each other.

STUDY NO. 1—ANALYSIS METHODOLOGY Means for cage weight gain, feed consumption, feed conversion, lesion scores, oocyst counts (OPGs), and mortality were calculated.

STUDY NO. 1—RESULTS Test results demonstrate that overall FCR was reduced in the compound treatment group compared to the challenged control group. Animals fed the inventive compound had similar FCR to the Salinomycin (medicated) treatment group.

Study No. 2

The second study to demonstrate the effectiveness of the compound of the disclosed inventive concept and a treatment method was designed to identify the comparative efficacy of the inventive compound administered in feed for the control of necrotic enteritis caused by Clostridium perfringens in animals, particularly chickens, and more specifically, broiler chickens. Clostridium perfringens is a spore forming anaerobe bacteria. It is commonly found in soil, dust, feed, poultry litter, feces, and in the gut.

Study No. 2—Treatment Method

An example of another treatment method using the inventive compound is set forth. It is to be understood that the following method is not intended as being the sole treatment method but is only exemplary. The study compared five treatment regimens: (1) no additive material in the feed, no coccidiosis/Clostridium perfringens challenge in the poultry, (2) no additive material in the feed, coccidiosis/Clostridium perfringens challenge in the poultry, (3) a compound inclusion rate of 0.039% in the feed, coccidiosis/Clostridium perfringens challenge in the poultry, (4) a compound inclusion rate of 0.077% in the feed, coccidiosis/Clostridium perfringens challenge in the poultry, and (5) a compound inclusion rate of 0.551% in the feed, coccidiosis/Clostridium perfringens challenge in the poultry. As set forth below, the study consisted of forty cages starting with nine chicks each. The treatments were replicated in eight blocks of five cages each. The chickens were 0 to 28 days of age.

An un-medicated chicken starter compounded with feedstuffs commonly used in the United States was formulated. The diet was representative of a local commercial formulation and calculated analyses met or exceeded broiler starter requirements of the US National Research Council (NRC). The diet formulation was included in the source data. Experimental treatment feeds were prepared from this basal starter feed. Quantities of all basal feed and test articles used to prepare treatment batches were documented. Treatment feeds were mixed to assure a uniform distribution of respective test article. The mixer was flushed to prevent cross contamination. The feed was distributed among cages of the same treatment. This ration (in mash form) was fed during the study. One each from the beginning, middle, and end of each batch of treatment diet was collected and mixed to form a composite sample.

Day of hatch male broiler chicks were obtained. Breeder flock information was recorded. At the hatchery, the birds were sexed. Only healthy appearing chicks were used in the study. Disposition of all birds not used for allocation was documented. There were 72 birds per treatment group.

Upon arrival, the chicks were raised in Petersime® battery cages. At placement the birds were fed the treatment feeds. Thermostatically controlled gas furnace/air conditioner maintained uniform temperature. Even illumination was provided. The cage diagram was documented. Cages were blocked by location in the battery. The study began when the birds were placed (day of hatch) (DOT 0) at which time they were allocated to the experimental cages. No birds were replaced during the course of the study.

All birds were weighed on DOT 0, 14, 21, and 28. Feed was weighed in on DOT 0 and the remaining feed was weighed on DOT 14, 21, and 28.

On DOT 14, all birds were orally inoculated with ˜5,000 oocysts of E. maxima. Starting on DOT 19, all birds, except Treatment 1 were given a broth culture of C. perfringens. The birds were administered a fresh broth culture once daily for 3 days (on DOTs 19, 20, and 21).

Study No. 2—Analysis Methodology

On DOT 21, three birds from each cage were selected, sacrificed, weighed, and examined for the degree of presence of necrotic cnteritis lesions. The scoring was based on a 0 to 3 score, with 0 being normal and 3 being the most severe. On DOT 28, one bird from each cage was selected, sacrificed, weighed, and the entire portion of the gastrointestinal tract was collected. On DOT 28, one bird from each cage was selected, euthanized, and a small cross section of intestine was cut and frozen. Means for cage weight gain, feed consumption, feed conversion, NE lesion scores, and the percent of necrotic enteritis mortality were calculated.

| | Coccidial | Clostridium | |
|---|---|---|---|
| | Challenge | perfringens | Cages/Trt |
| 1.Nonmedicated | DOT 14 | No | 8 |
| 2. Nonmedicated | DOT 14 | DOT 19, 20, and 21 | 8 |
| 3. Compound inclusion rate | DOT 14 | DOT 19, 20, and 21 | 8 |
| 4. Compound inclusion rate | DOT 14 | DOT 19, 20, and 21 | 8 |
| 5. Compound inclusion rate | DOT 14 | DOT 19, 20, and 21 | 8 |

Study No. 2—Collected Data

Charts setting forth the collected data follow.

| | Feed | Feed | Feed | Feed | Weight | Weight |
|---|---|---|---|---|---|---|
| | Intake | Intake | Conversion | Conversion | Gain | Gain |
| Treatment | D0-21 | D14-21 | D0-21 | D14-21 | D0-21 | D14-21 |
| 1. NM , No | 7.053a | 3.971a | 1.516b | 1.440b | 0.532a | 0.319a |
| CP | | | | | | |
| 2. NM, CP | 7.297a | 3.654a | 1.725a | 1.745a | 0.477b | 0.240c |
| 3. Inventive | 7.213a | 3.840a | 1.561b | 1.586b | 0.527a | 0.279b |
| Compound | | | | | | |
| 0.039%, CP | | | | | | |
| 4. Inventive | 6.820a | 3.651a | 1.557b | 1.594ab | 0.510ab | 0.283b |
| Compound | | | | | | |
| 077%, CP | | | | | | |
| 5. Inventive | 6.985a | 3.815a | 1.510b | 1.537b | 0.526a | 0.268b |
| Compound | | | | | | |
| 0.551%, CP | | | | | | |
| | Feed | Feed | Feed | Feed | Weight | Weight |
| | Intake | Intake | Conversion | Conversion | Gain | Gain |
| Treatment | D0-28 | D14-28 | D0-28 | D14-28 | D0-28 | D14-28 |
| 1. NM , No | 9.570a | 6.488a | 1.540b | 1.501c | 0.777a | 0.564a |
| CP | | | | | | |
| 2. NM, CP | 9.113a | 5.470c | 1.753a | 1.787a | 0.731a | 0.494a |
| 3. Inventive | 9.320a | 5. 948abc | 1.609b | 1.656b | 0.794a | 0.546a |
| Compound | | | | | | |
| 0.039%, CP | | | | | | |
| 4. Inventive | 9.005a | 5.837bc | 1.588b | 1.637b | 0.762a | 0.520a |
| Compound | | | | | | |
| 0.077%, CP | | | | | | |
| 5. Inventive | 9.553a | 6.384ab | 1.589b | 1.646b | 0.765a | 0.522a |
| Compound | | | | | | |
| 0.551%, CP | | | | | | |

| Treatment | Score NE (0-3) | Mortality % NE |
|---|---|---|
| 1.NM, No CP | 0.0b | 0d |
| 2.NM, CP | 1.1a | 25a |
| 3.Inventive Compound 0.039%, CP | 0.8a | 13b |
| 4.Inventive Compound 0.077%, CP | 0.9a | 9bc |
| 5.Inventive Compound 0.551%, CP | 0.9a | 5cd |

The letters shown with each result denote statistical significance—those with the same letters are not statistically different from each other.

STUDY NO. 2—RESULTS Test results demonstrate that overall FCR was reduced in the compound treatment group compared to the challenged control group. Animals fed the inventive compound had similar FCR to the unchallenged control group. Percent necrotic enteritis mortality was significantly reduced with birds fed the inventive compound.

Study No. 3

The third study to demonstrate the effectiveness of the compound of the disclosed inventive concept and a treatment method was designed to identify the comparative efficacy on broiler performance and digestive health of the inventive compound administered to floor-pen raised broilers in a normal formulated Corn/SBM diet, without coccidiostat or other ABF products, and reared under a disease challenge environment (cocci-challenge+built-up litter).

Study No. 3—Treatment Method

A non-limiting example of another treatment method using the inventive compound is set forth. It is to be understood that the following method is not intended as being the sole treatment method but is only exemplary. The study compared five treatment regimens: (1) no additive material in the feed, no coccidiosis challenge in the poultry, (2) no additive material in the feed, coccidiosis challenge in the poultry, (3) Coban (9 g/ton of feed), coccidiosis challenge in the poultry, and (4) the inventive compound in the feed at an inclusion rate of 125 g/ton of feed, coccidiosis challenge in the poultry. Coban® (Monensin, USP) is commonly used as an antibiotic in the treatment of coccidiosis.

The study animals were mixed-sex commercial broiler chicks obtained within 12-hrs of hatching from fecal contaminated flocks at a commercial hatchery. No coccidiosis vaccine was administered at the hatchery or at any time during the study. Chicks were transported to research pens under temperature-controlled conditions to assure bird comfort. Upon arrival, chicks were immediately randomly assigned to each experimental pen.

Broiler chickens were placed in pens containing built-up litter floor bedding, from at least 3-previous flocks. Pens measured 4.5′×10′ to provide approximately 0.87 ft2 per bird. Initially (Days 0-10), the birds were placed on a partial house brooding system (about 0.45 ft2 per bird). There were 240 birds per treatment group.

Birds were fed a standard Corn-SBM diet with normal nutritional formulations. Feed was weighed at the beginning of each formulation period and fed in two phases: Starter diet (0-14 days of age), Grower diet (15-28 days of age).

On Trial Day 7, all birds in the challenged groups received oocyst-inoculated feed containing a mixture of three Eimeria species. Adequate feed was precisely weighed and provided to birds to consume at the rate of 100% fill-capacity on average, which was determined by measuring the quantity of feed consumed within a 24-hr period the previous day for each pen. Oocyst were mixed with feed for 10 minutes using a 50# mixer to provide 100,000 oocysts per bird E. acervuline, 50,000 oocysts per bird E. maxima, and 75,000 oocysts per bird E. tenella. Prior to challenge, all cocci-inoculated birds were starved for 8 hours. Inoculated feed was placed on each block (or rep) at the same time and allowed to remain for 2 hours. Following the 2-hr program, all remaining inoculated feed was removed and weighed to assure equal consumption per pen and per bird.

Study No. 3—Analysis Methodology

Measurement endpoints were taken for each treatment group for growth live performance, which included mortality, feed intake, weight gains following each period and feed:gain values (i.e., feed conversion ratio) on days 0-14, 0-21, and 0-28 days.

On day 14, gut duodenum lesion scores and Coccidiosis/Eimeria ceca lesion scores were taken on 2-males and 2-females per pen and on 28 days of age from 4-males and 4-females per pen.

Study No. 3—Data

The collected data are set forth in the following chart.

| | | Avg Body | | |
|---|---|---|---|---|
| | Feed Intake | Weight | | |
| DAY 0-14 | (g/bird/day) | (g) | FCR | % Mortality |
| 1. No Additive, Non-Infected | 33.246a | 481.68b | 1.090a | 0.417a |
| 2. No Additive, Infected | 32.790a | 450.52c | 1.142b | 3.333b |
| 3. Coban 90 g/ton | 34.680b | 496.13a | 1.112ab | 0.833a |
| 4. Inventive Compound 125 g/ton | 34.713b | 483.01b | 1.129b | 1.250a |
| | | Avg Body | | |
| | Feed Intake | Weight | | |
| DAY 15-21 | (g/bird/day) | (g) | FCR | % Mortality |
| 1. No Additive, Non-Infected | 76.237a | 432.825a | 1.239ab | 0.625a |
| 2. No Additive, Infected | 72.484a | 385.175b | 1.323b | 4.479b |
| 3. Coban 90 g/ton | 77.988a | 445.167a | 1.244ab | 0.729a |
| 4. Inventive Compound 125 g/ton | 74.492a | 428.125a | 1.208a | 1.354a |
| | | Avg Body | | |
| | Feed Intake | Weight | | |
| DAY 22-28 | (g/bird/day) | (g) | FCR | % Mortality |
| 1. No Additive, Non-Infected | 138.354b | 537.183a | 1.805a | 0.000a |
| 2. No Additive, Infected | 121.443a | 452.450b | 1.888a | 1.042a |
| 3. Coban 90 g/ton | 151.410c | 583.217a | 1.825a | 0.000a |
| 4. Inventive Compound 125 g/ton | 142.092bc | 555.683a | 1.794a | 0.000a |
| DAY 14 Avg Lesion Score | Duodenum | | Ceca | |
| 1. No Additive, Non-Infected | 0.125a | | 0.146a | |
| 2. No Additive, Infected | 1.583d | | 1.729c | |
| 3. Coban 90 g/ton | 0.375b | | 0.292a | |
| 4. Inventive Compound 125 g/ton | 0.646c | | 0.667b | |
| DAY 28 Avg Lesion Score | Duodenum | | Ceca | |
| 1. No Additive, Non-Infected | 0.167a | | 0.188a | |
| 2. No Additive, Infected | 1.563c | | 1.667c | |
| 3. Coban 90 g/ton | 0.208a | | 0.229a | |
| 4. Inventive Compound 125 g/ton | 0.625b | | 0.563b | |
| | | Avg Body | | |
| | Feed Intake | Weight | | |
| DAY 0-14 | (g/bird/day) | (g) | FCR | % Mortality |
| 1. No Additive, Non-Infected | 33.246a | 481.68b | 1.090a | 0.417a |
| 2. No Additive, Infected | 32.790a | 450.52c | 1.142b | 3.333b |
| 3. Coban 90 g/ton | 34.680b | 496.13a | 1.112ab | 0.833a |
| 4. Inventive Compound 125 g/ton | 34.713b | 483.01b | 1.129b | 1.250a |
| | | Avg Body | | |
| | Feed Intake | Weight | | |
| DAY 0-21 | (g/bird/day) | (g) | FCR | % Mortality |
| 1. No Additive, Non-Infected | 53.309a | 914.50b | 1.155a | 1.042a |
| 2. No Additive, Infected | 52.848a | 835.70c | 1.217b | 7.813b |
| 3. Coban 90 g/ton | 54.811a | 951.80a | 1.151a | 1.563a |
| 4. Inventive Compound 125 g/ton | 54.198a | 911.10b | 1.165a | 2.604a |
| | | Avg Body | | |
| | Feed Intake | Weight | | |
| DAY 0-28 | (g/bird/day) | (g) | FCR | % Mortality |
| 1. No Additive, Non-Infected | 74.570b | 1451.70b | 1.385a | 1.042a |
| 2. No Additive, Infected | 70.520a | 1288.10c | 1.434b | 8.854b |
| 3. Coban 90 g/ton | 78.960c | 1535.00a | 1.387a | 1.563a |
| 4. Inventive Compound 125 g/ton | 76.171bc | 1466.80b | 1.392a | 2.604a |

The letters shown with each result denote statistical significance—those with the same letters are not statistically different from each other.

STUDY NO. 3—RESULTS Test results demonstrate that overall FCR was reduced in the compound treatment group compared to the challenged control group beginning at Day 15. Animals fed the inventive compound had the lowest numerical FCR of all groups from Day 15 to the end of the study (Day 28). The average body weight of the animals fed the inventive compound were not statistically different from the non-infected control group throughout the study. Mortality was significantly reduced as well, and not statistically different from the non-infected control group or the Coban-treated group.

What is claimed is:

1. A method for mitigating coccidial infection in an animal, the method comprising the modulation of the Toll-like receptor pathway by feeding to the animal an effective amount of a composition derived from a biomass comprising a lipopolysaccharide derived from Gram-negative bacteria, wherein the effects of coccidiosis are mitigated via an enhanced immune response due to broad spectrum immune modulation.

2. The method of claim 1, whereby the composition is mixed with a feed ration portion prior to feeding the animal.

3. The method of claim 2, where the Gram-negative bacteria is from the genus Rhodobacter or Variovorax.

4. The method of claim 1, wherein said Gram-negative bacteria is a member of the group Variovorax.

5. The method of claim 4, wherein said member of the group Variovorax is Variovorax paradoxus.

6. The method of claim 2, wherein the composition derived from the biomass consisting of Gram-negative bacteria is fed to the animal in an amount providing from about 20.0 g composition per ton of finished feed to about 250.0 g composition per ton of finished feed.

7. The method of claim 2, wherein the composition derived from the biomass consisting of Gram-negative bacteria is fed to the animal in an amount providing from about 125.0 g composition per ton of finished feed to about 175.0 g composition per ton of finished feed.

8. The method of claim 2, wherein the composition derived from the biomass consisting of Gram-negative bacteria is fed to the animal in an amount providing from about 100.0 g composition per ton of finished feed to about 150.0 g composition per ton of finished feed.

9. The method of claim 1, wherein the composition comprising the lipopolysaccharide derived from Gram-negative bacteria composition is adapted for use in poultry.

10. The method of claim 1, wherein the composition comprising the lipopolysaccharide derived from Gram-negative bacteria composition is for the mitigation of coccidiosis in poultry.

11. A method of mitigating coccidiosis in an animal, the method comprising the step of modulating the Toll-like receptor pathway by feeding to the animal an effective amount of a composition derived from a biomass comprising a lipopolysaccharide derived from Gram-negative bacteria, the composition being fed to the animal in an amount of from about 20.0 g composition per ton of finished feed to about 250.0 g composition per ton of finished feed.

12. The method of claim 11, wherein the composition derived from the biomass consisting of Gram-negative bacteria is fed to the animal in an amount providing from about 125.0 g composition per ton of finished feed to about 175.0 g composition per ton of finished feed.

13. The method of claim 11, wherein the composition derived from the biomass consisting of Gram-negative bacteria is fed to the animal in an amount providing from about 100.0 g composition per ton of finished feed to about 150.0 g composition per ton of finished feed.

14. The method of claim 11, where the Gram-negative bacteria is from the genus Rhodobacter or Variovorax.

15. The method of claim 11, wherein said Gram-negative bacteria is a member of the group Variovorax.

16. The method of claim 15, wherein said member of the group Variovorax is Variovorax paradoxus.

17. The method of claim 11, wherein the composition comprising the lipopolysaccharide derived from Gram-negative bacteria composition is adapted for use in poultry.

18. The method of claim 11, wherein the composition comprising the lipopolysaccharide derived from Gram-negative bacteria composition is for the mitigation of coccidiosis in poultry.

19. A method for preventing or minimizing diseases which cause gut inflammation in animals, the method comprising creating a direct effect on innate and adaptive immune pathways by feeding to the animal an effective amount of a composition comprising a lipopolysaccharide derived from Gram-negative bacteria, wherein the effects of the gut inflaming disease are mitigated via an enhanced immune response.

20. The method of claim 19, wherein the composition consisting of Gram-negative bacteria is fed to the animal in an amount providing from about 20.0 g composition per ton of finished feed to about 250.0 g composition per ton of finished feed.
